(19) United States
(12) Patent Application Publication
(10) Pub. No.: US 2009/0326828 A1
(43) Pub. Date: Dec. 31, 2009

(54) Optimizing Selection of SRM Transitions for Analysis of Biomolecules by Tandem Mass Spectrometry

(76) Inventor: Amol Prakash, Cambridge, MA (US)

Correspondence Address: THERMO FINNIGAN LLC, 355 RIVER OAKS PARKWAY, SAN JOSE, CA, 95134, US

(21) Appl. No.: 12/163,928

(22) Filed: Jun. 27, 2008

(51) Int. Cl. G01N 33/68

(52) U.S. Cl. 702/19

(57) ABSTRACT

Methods for selecting a set of SRM transitions for a peptide of interest include selecting a first transition based on sensitivity criteria and selecting at least a second transition based on selectivity criteria. A determination of the uniqueness of the first transition combined with the at least a second transition is made. When the combination of the first transition and the at least a second transition is determined to be unique to the peptide of interest, a sample containing the peptide of interest is subjected to a SRM workflow by monitoring the first transition and the second transition. Also described is an apparatus for carrying out the methods.

### FIELD OF THE INVENTION

The instant invention relates generally to assays for proteins/peptides based on mass spectrometry by selective reaction monitoring (SRM), and more particularly to a method and system for selecting SRM transitions for a peptide of interest based on both selectivity criteria and sensitivity criteria.

### BACKGROUND OF THE INVENTION

Mass spectrometry-based quantitative proteomics has become an important component of biological and clinical research. In common mass spectrometry (MS)-based proteomic pipelines, protein samples are first partially purified or separated by chromatographic or electrophoretic methods and then digested with a proteolytic enzyme such as trypsin, often resulting in highly complex peptide mixtures. These mixtures are further separated by one or more stages of capillary liquid chromatography (LC) and analyzed using a tandem mass spectrometer.

One common mass spectrometric approach is selective reaction monitoring (SRM) based targeted discovery. Starting with a peptide of interest, candidate fragment ions are chosen to set mass-to-charge (m/z) values in the Q1/Q3 cells of a triple quadrupole mass spectrometer. As the sample flows through the LC column and is introduced into the mass spectrometer, ions having the specified m/z's are transmitted through the Q1 and Q3 cells and are detected. Unfortunately, in a complex background like plasma various analytes can yield signals that approximate the peptide of interest, thereby leading to false positives. Accordingly, a highly selective list of transitions defining a unique signature for the peptide of interest is required in order to achieve confident identification and quantification of that peptide. However, on the sensitivity side one also needs to monitor the most intense transitions, so as to have the most sensitive assay for quantifying low abundance proteins. Typically, the process of building selective and sensitive assays is very challenging and requires large amounts of samples and multiple iterations.

Accordingly, there exists a need for a method that overcomes at least some of the above-mentioned limitations.

### SUMMARY OF EMBODIMENTS OF THE INVENTION

According to an aspect of the instant invention there is provided a method for selecting a set of transitions for a peptide of interest, the set of transitions for identifying uniquely the peptide of interest in a Selective Reaction Monitoring (SRM) workflow, the method comprising: selecting from a plurality of candidate transitions for the peptide of interest a first transition, the first transition being the expected most sensitive transition for the peptide of interest; selecting from the plurality of candidate transitions for the peptide of interest at least a second transition, the selection of the at least a second transition being performed in an iterative fashion until the set of transitions consisting of the first transition and the at least a second transition is determined to be unique to the peptide of interest; and, subjecting to the SRM workflow a sample including the peptide of interest, comprising monitoring the first transition and the at least a second transition for at least one of identifying and quantifying the peptide of interest.

According to an aspect of the instant invention there is provided a method for selecting a set of transitions for a peptide of interest, the set of transitions for identifying uniquely the peptide of interest in a Selective Reaction Monitoring (SRM) workflow, the method comprising: displaying to a user data that are indicative of a plurality of candidate transitions for the peptide of interest; receiving from the user a first input signal for selecting a first transition from the plurality of candidate transitions, the first transition being selected on the basis of sensitivity criteria; and, indicating to the user at least a second transition of the plurality of candidate transitions which, in combination with the first transition of the plurality of candidate transitions, defines the set of transitions for identifying uniquely the peptide of interest.

According to an aspect of the instant invention there is provided a system for selecting a set of transitions for a peptide of interest, the set of transitions for identifying uniquely the peptide of interest in a Selective Reaction Monitoring (SRM) workflow, the system comprising: a controller for identifying a first SRM transition for the peptide of interest on the basis of sensitivity criteria and for identifying at least a second SRM transition for the peptide of interest on the basis of selectivity criteria, the first SRM transition relating to the formation of a first type of product ion by fragmentation of the peptide of interest and the at least a second SRM transition relating to the formation of at least a second type of product ion by fragmentation of the peptide of interest; a detector; a first mass separator for transmitting ions having a mass-to-charge (m/z) ratio corresponding to the peptide of interest; an ion fragmentor for receiving the ions from the first mass separator and for fragmenting the ions to form a plurality of types of product ions; and, a second mass separator for transmitting to the detector, sequentially in time, the first type of product ion and each different product ion type of the at least a second type of product ion.

### BRIEF DESCRIPTION OF THE DRAWINGS

Exemplary embodiments of the invention will now be described in conjunction with the following drawings, in which similar reference numerals designate similar items:

FIG. 1 is a simplified schematic diagram of a mass spectrometer system that is suitable for performing selective reaction monitoring (SRM) experiments;

FIG. 2 is a schematic view of a triple quadrupole mass spectrometer system including an atmospheric pressure ion source coupled to a tandem mass analyzer through evacuated interface chambers with multipole ion guides;

FIG. 3 is a simplified flow diagram of a method according to an embodiment of the instant invention;

FIG. 4 is a simplified flow diagram of a method according to an embodiment of the instant invention; and,

FIG. 5 is a simplified flow diagram of a method according to an embodiment of the instant invention.

### DETAILED DESCRIPTION OF EMBODIMENTS OF THE INSTANT INVENTION

The following description is presented to enable a person skilled in the art to make and use the invention, and is provided in the context of a particular application and its requirements. Various modifications to the disclosed embodiments will be readily apparent to those skilled in the art, and the general principles defined herein may be applied to other embodiments and applications. Thus, the present invention is not intended to be limited to the embodiments disclosed, but is to be accorded the widest scope consistent with the principles and features disclosed herein.

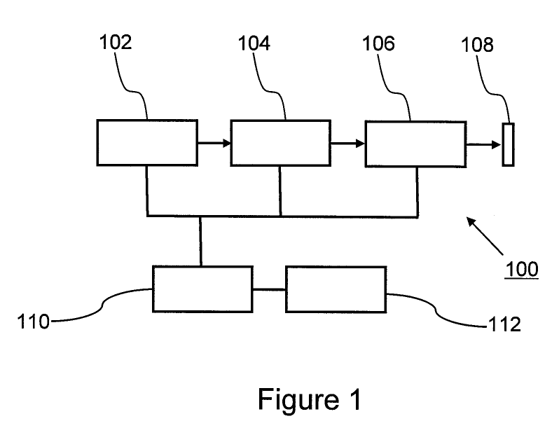

Referring to FIG. 1, shown is a simplified schematic diagram of a mass spectrometer system 100 that is suitable for performing SRM experiments. A SRM scan is obtained by setting a first mass separator 102 to transmit ions in a narrow m/z window that is centered on the m/z value of a molecular ion of a peptide of interest to an ion fragmentor, such as for instance collision cell 104. By way of a specific and non-limiting example, the collision cell comprises a collision gas for conducting collision-induced dissociation of the peptide of interest and a quadrupole to facilitate radial confinement and transmittal of the fragment ions. Furthermore, the collision energy of collision cell 104 can be selected to facilitate producing desired fragment ions of the peptide of interest. The second mass separator 106 is set to transmit to a detector 108, ions in an m/z window that is centered on the m/z value of a specified fragment ion so as to generate a signal representative of the abundance of the specified fragment ions.

Also shown in FIG. 1 is a controller 110 for use in selecting SRM transitions for a peptide of interest based on both selectivity criteria and sensitivity criteria, according to an embodiment of the instant invention. The controller 110 is in communication with a memory storage device 112. The memory storage device 112 has retrievably stored thereon an information database, which comprises information relating to transitions for unmodified and/or modified peptides. Such database libraries are available commercially or may be custom generated to suit specific needs. The controller 110 uses the information that is stored in the database for “digesting” the sample matrix in silico, as discussed in greater detail below. Optionally, a not illustrated user interface comprising a display device and a data input device is provided in communication with the controller 110. During implementation of a semi-automated method according to an embodiment of the instant invention, a user uses the input device to select one or more SRM transitions from a displayed list.

One specific and non-limiting example of a suitable mass spectrometer system is a conventional triple quadrupole mass spectrometer, including two quadrupole mass filters with a collision cell disposed in the ion flight path therebetween. Other structures capable of performing the mass filtering and dissociation functions may be substituted for the quadrupole mass filters and collision cell, respectively.

A suitable ion source (not shown) for the mass spectrometer system of FIG. 1 includes, but is not limited to, an electrospray ionization (ESI) or atmospheric pressure chemical ionization (APCI) source. For example, an ESI ion source supports introduction of an ionized sample that originates from a liquid chromatography column into a mass separator apparatus. One of several desirable features of ESI is that fractions of the eluate from the chromatography column can proceed directly from the column to the ESI ion source.

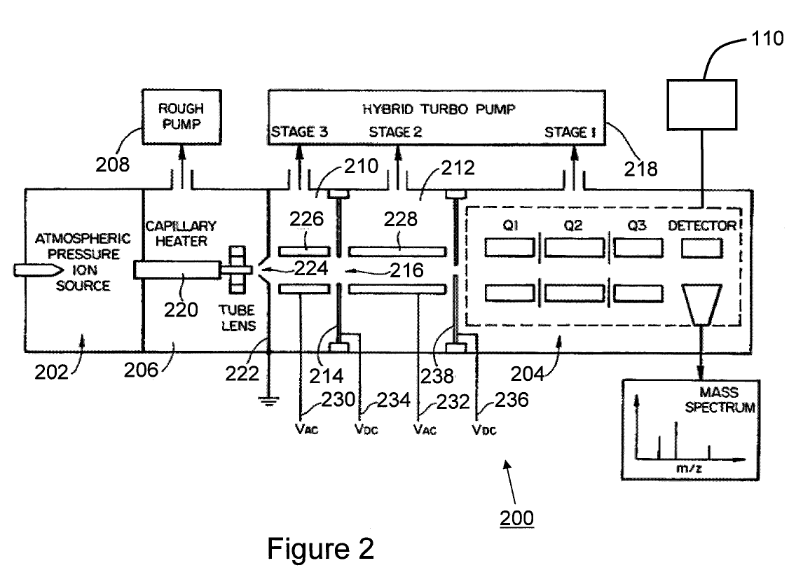

Referring now to FIG. 2, shown is a schematic diagram of a triple quadrupole mass spectrometer system 200, which is suitable for performing SRM experiments. An atmospheric pressure ion source in chamber 202 is interfaced to a tandem mass analyzer 204 via three vacuum pumping stages. The first stage 206, which has the highest pressure, is evacuated by an oil-filled rotary vane vacuum pump 208. Other types of vacuum pumps may also be used for this stage, such as a diaphragm pump or scroll pump. The second and third stages 210 and 212 are separated by a lens 214 with an orifice 216, and can be evacuated by a hybrid or compound turbomolecular pump 218 which includes both turbomolecular and molecular drag pumping stages, and may have multiple inlets into each of these pumping stages, or by individual vacuum pumps (not shown).

The atmospheric pressure ion source may be an electrospray ion source or atmospheric pressure chemical ionization source. With either ion source, sample liquid is introduced into the chamber 202, which is at atmospheric pressure, and ionized. The ions are drawn through a capillary 220, which may be heated, into chamber 206. The end of the capillary is opposite a conical skimmer 222, which includes a central orifice or aperture 224. The skimmer separates the low pressure stage 206 from the lower pressure stage 210. A portion of the ion and gas flow is skimmed from the free jet expansion leaving the capillary and enters the second lower pressure stage. The ions that travel through the skimmer are guided into the mass analyzer by first and second multipole ion guides 226 and 228. The quadrupole ion guides are operated by applying AC voltages 230 and 232 in a prescribed phase relationship to the rod electrodes to radially confine ions in a known manner. Ions which enter the second and third stages drift under the influence of DC voltage 234 applied between the skimmer lens 222 and lens 214, by DC voltage 236 applied between the lens 214 and the lens 238, and by DC offset voltages applied to ion guides 226 and 228.

Referring still to FIG. 2, in a SRM experiment a sample containing a peptide of interest is loaded onto a not illustrated liquid chromatography column, and a not illustrated pump is used to produce and deliver a solvent gradient to the column. A portion of the eluate from the liquid chromatography column is introduced into the atmospheric pressure ion source in chamber 202 to produce ions from the sample, which ions subsequently are introduced into the tandem mass analyzer 204. The Q1 of the tandem mass analyzer 204 is set (by appropriately adjusting the radio frequency and DC filtering voltages applied to the Q1 electrodes) to the m/z value of the peptide of interest, and the Q3 of the tandem mass analyzer 204 is set, sequentially in time, to the m/z value of each of a plurality of candidate fragment ion m/z values (by appropriately adjusting the radio frequency and DC filtering voltages applied to the Q3 electrodes), so as to measure the ion signal resulting for each transition corresponding to the plurality of fragment ion m/z values. A plot of the measured ion signals versus time yields a chromatogram for each of the candidate fragment ions.

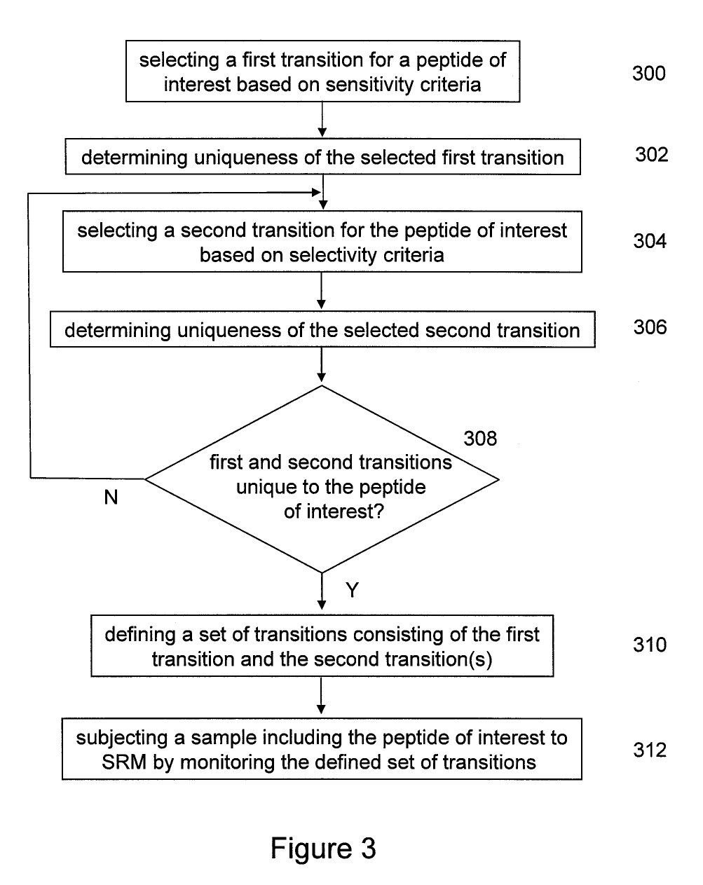

Referring to FIGS. 3 through 5, shown are methods according to various embodiments of the instant invention for selecting, based on separate selectivity criteria and sensitivity criteria, SRM transitions for a peptide of interest. For the purpose of discussion, it is assumed that a triple quadrupole mass spectrometer is used. However, in practice any suitable mass spectrometer system may be used to implement the methods that are discussed with reference to FIGS. 3 through 5. Furthermore, the methods are implemented optionally in either a semi-automated or an automated fashion.

Referring now to FIG. 3, for the case of a semi-automated implementation, a user begins by selecting at step 300 a first transition for a peptide of interest based on sensitivity criteria. For instance, the user selects a most sensitive transition for the peptide of interest. The selection of the first transition is based on, for instance, previous knowledge or historical data relating to the peptide of interest. Optionally, the selection of the first transition is based on the application of known chemistry rules, so as to determine an expected most sensitive transition. The subsequent application of fragmentation chemistry rules can then help guide the choice of the most sensitive transition(s). Further optionally, the first transition is selected in a pseudo-random fashion from a plurality of known sensitive transitions for the peptide of interest. Of course, in an automated implementation the first transition for a peptide of interest is selected absent user intervention. Furthermore, the first transition optionally is a set of transitions comprising a plurality of transitions, each one of the plurality of transitions being selected based on sensitivity criteria.

Once it has been selected, the uniqueness of the first transition is then determined at step 302. More specifically, the sample matrix is “digested” in silico in order to determine the number of possible interferences that may arise from the sample matrix. As will be apparent to a person having ordinary skill in the art, a complex matrix such as human plasma or mouse tissue contains hundreds of different proteins. Upon digestion, each protein yields tens or even hundreds of peptide fragments, each of which may in turn exist in a plurality of different charge states, and each of which may additionally fragment to form a plurality of different product ions. Due to the very large number of ions that may be observed when the sample matrix is ionized, the likelihood of there being one or more interference with the first transition is high.

In order to “digest” the sample matrix in silico, the controller 110 of the mass spectrometer system utilizes the database that is stored on memory storage device 112, and determines the maximum number of interferences with the selected first transition. The determination is performed taking into account the operating parameters of the mass spectrometer system. For instance, the resolution of the mass spectrometer system affects the number of possible interferences for a given sample. Under otherwise identical operating conditions a lower resolution results in more interferences being identified than does a higher resolution. In addition, other factors such as for instance the proteolytic enzyme used must also be considered. Optionally, the uniqueness determination also takes into account other operating parameters, such as for instance the effect of sample separation by liquid chromatography prior to ionization. For instance those peptides that elute at different times than the peptide of interest, due to differences in the hydrophobicity thereof, may be disregarded during the uniqueness determination. Furthermore, peptides that are not ionized to a significant extent or that are known to be of low abundance in the sample may be excluded from the list of possible interferences with the selected first transition. For instance those peptides having a relative abundance below a predetermined threshold value are disregarded during the uniqueness determination.

Once the uniqueness of the selected first transition is determined, the controller 110 “suggests” a second transition at step 304, in order to define a set of transitions that is unique to the peptide of interest. The selection of the second transition is based on selectivity criteria. For instance, at step 306 the uniqueness of the suggested second transition is determined in a manner similar to that which was described above with reference to the selected first transition. If it is determined at step 308 that a set of transitions consisting of the selected first transition and the suggested second transition is unique to the peptide of interest, then no additional second transitions need to be selected. The set of transitions is defined at step 310, and at step 312 a sample including the peptide of interest is subjected to SRM by monitoring the first transition and the second transition(s). However, if the set is not unique to the peptide of interest, then the steps 304 through 308 are repeated and either an additional second transition is suggested for being added to the set, or a different second transition is suggested. Once the first and second transitions are determined to be unique to the peptide of interest, the steps 310 and 312 are performed as discussed supra. Accordingly, the selection of a set of transitions that is unique to the peptide of interest, based upon the determination of potential interferences arising from the sample matrix following in silico digestion, proceeds in an iterative manner. While it is not an essential feature of the instant invention, typically the suggested second transition is selected from a sub-group of transitions in the neighborhood of the selected first transition, as is discussed below in greater detail.

In order to facilitate a better understanding of the principles that have been outlined above, a specific and non-limiting example will now be provided using the peptide SSFVALELEK. In this case the transitions are selected for the +2 charge state of the peptide of interest, such that the m/z value of the precursor ion is 561.806 and the product ions are singly charged. Instruction code, or software, that is in execution on a processor of the controller 110 (see FIG. 2) causes data relating to the precursor ion to be displayed to a user. A list of the possible product ions, both the b-series ions and the y-series ions, also is displayed to the user. Furthermore, one of the product ions is highlighted or otherwise identified to the user as satisfying sensitivity criteria for the peptide of interest. For instance, based on fragmentation chemistry rules the software suggests to the user selecting the y6 ion (m/z 702.403) as the most sensitive ion. Once the user selects the y6 ion, the software determines the uniqueness of the selected transition (m/z 561.806→702.403). In the instant example the software determines that 53 other proteins in the Human IPI database have a transition that interferes with the selected transition, within the instrument resolution. Accordingly, the software suggests the y7 ion as the next most selective ion. Once the user selects the y7 ion, the software determines the uniqueness of the selected transition (m/z 561.806→801.471). The software determines that 38 other proteins in the Human IPI database have a transition that interferes with the latter transition, but that no other proteins in the Human IPI database have transitions that match the combination of the two transitions. Accordingly, a set of transitions including the y6 and y7 ions is unique for the peptide of interest, and additionally satisfies sensitivity criteria. As noted above, the suggested second transition (precursor→y7) is in the neighborhood of the first transition (precursor→y6).

In contrast, had the user instead selected the y5 ion for the second transition, then the uniqueness determination would have revealed that 52 other proteins in the Human IPI database have a transition that interferes therewith, and that 7 other proteins in the Human IPI database have transitions that match the combination of the two transitions. Accordingly, a set of transitions including only the y6 and the y5 ions is not unique for the peptide of interest. In this case, optionally a different ion is suggested for the second transition or an additional ion is selected in order to produce a set of transitions that is unique for the peptide of interest.

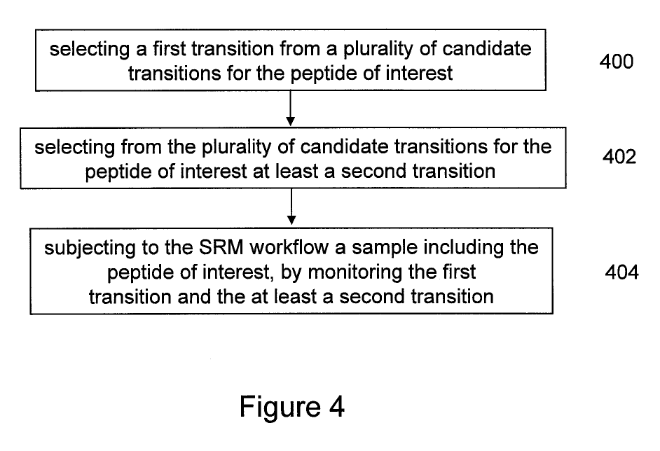

Referring now to FIG. 4, shown is a simplified flow diagram of a method according to an embodiment of the instant invention. At 400 a first transition is selected from a plurality of candidate transitions for the peptide of interest. For instance, the selected first transition is the expected most sensitive transition for the peptide of interest. Optionally, the first transition is a set of transitions comprising a plurality of transitions, each one of the plurality of transitions being selected based on sensitivity criteria. At 402 at least a second transition is selected from the plurality of candidate transitions for the peptide of interest. The selection of the at least a second transition is performed in an iterative fashion, until the set of transitions consisting of the first transition and the at least a second transition is determined to be unique to the peptide of interest. At 404 a sample including the peptide of interest is subjected to the SRM workflow, whereby the first transition and the at least a second transition are monitored for at least one of identifying and quantifying the peptide of interest.

As discussed above, optionally the first transition is selected based on previous knowledge relating to the peptide of interest. One possibility can be the fragmentation pattern observed in a different setting (possibly a previous experiment or workflow on possibly a different instrument). Further optionally, the selection of the first transition is based on the application of known chemistry rules, or the first transition is selected in a pseudo-random fashion from a plurality of known sensitive transitions for the peptide of interest.

In a semi-automated implementation of the method of FIG. 4, a user selects the first transition and the at least a second transition using a user interface in communication with the controller 110. For instance, the controller 110 displays to the user a list of transitions for the peptide of interest, one of which is highlighted or otherwise indicated as being the most sensitive transition for the peptide of interest. Using a data input device, the user selects the indicated transition. The controller then indicates at least a second transition from the list of transitions for the peptide of interest. The user once again uses the data input device to select the indicated transition. However, if the user so wishes then one of the non-indicated transitions may be selected instead. This may occur when the user has previous experience with the peptide of interest. In this case, the controller bases all subsequent determinations on the user selected transition, rather than the originally indicated transition.

In an automated implementation of the method of FIG. 4, the transitions are selected absent user intervention. However, during initial set-up a user may specify in advance certain threshold values relating to the sensitivity criteria and the selectivity criteria. Furthermore, the user may be required to accept the final set of transitions as selected by the controller 110.

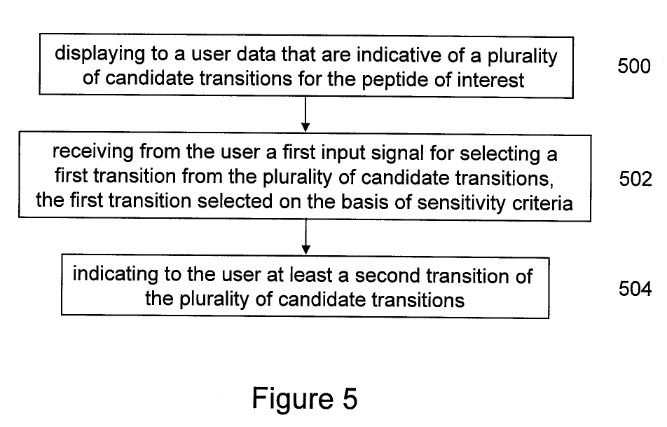

Referring now to FIG. 5, shown is a simplified flow diagram of a method according to an embodiment of the instant invention. At 500 data are displayed to a user, the data being indicative of a plurality of candidate transitions for the peptide of interest. At 502 a first input signal for selecting a first transition from the plurality of candidate transitions is received from the user. The first transition is selected on the basis of sensitivity criteria. Optionally, the first transition is a set of transitions comprising a plurality of transitions, each one of the plurality of transitions being selected based on sensitivity criteria. At 504 an indication is provided to the user for suggesting at least a second transition of the plurality of candidate transitions, which in combination with the first transition of the plurality of candidate transitions defines a set of transitions for identifying uniquely the peptide of interest.

Optionally, the methods according to the embodiments of the instant invention are performed in an automated manner, absent user intervention. In an automated implementation, the controller 110 yields a set of transitions including zero, one or more transitions for a peptide of interest as selected on the basis of sensitivity criteria. Additionally, the controller 110 yields a set of transitions as selected on the basis of selectivity criteria. In some cases, only a single transition may be required in order to satisfy both of the sensitivity criteria and the selectivity criteria. Optionally, a user is required to confirm acceptance of a set of transitions that is determined in an otherwise automated manner.

What is claimed is:

1. A method for selecting a set of transitions for identifying a set of transitions for a peptide of interest, comprising:
selecting from a plurality of candidate transitions for the peptide of interest a first transition, the first transition being the expected most sensitive transition for the peptide of interest;
selecting from the plurality of candidate transitions for the peptide of interest at least a second transition, the selection of the at least a second transition being performed in an iterative fashion until the set of transitions consisting of the first transition and the at least a second transition is determined to be unique to the peptide of interest; and,
subjecting to the SRM workflow a sample including the peptide of interest, comprising monitoring the first transition and the at least a second transition for at least one of identifying and quantifying the peptide of interest.

2. A method according to claim 1, comprising determining separately a uniqueness of each the first transition and of the at least a second transition.

3. A method according to claim 2, comprising determining an overall uniqueness of the set of transitions consisting of the first transition and the at least a second transition.

4. A method according to claim 1, wherein the at least a second transition comprises a plurality of different transitions.

5. A method according to claim 1, wherein the first transition is selected based on historical data for the peptide of interest.

6. A method according to claim 1, wherein the first transition is selected based on the application of a set of known fragmentation chemistry rules.

7. A method for selecting a set of transitions for a peptide of interest, the set of transitions for identifying uniquely the peptide of interest in a Selective Reaction Monitoring (SRM) workflow, the method comprising:
displaying to a user data that are indicative of a plurality of candidate transitions for the peptide of interest;
receiving from the user a first input signal for selecting a first transition from the plurality of candidate transitions, the first transition being selected on the basis of sensitivity criteria; and,
indicating to the user at least a second transition of the plurality of candidate transitions which, in combination with the first transition of the plurality of candidate transitions, defines the set of transitions for identifying uniquely the peptide of interest.

8. A method according to claim 7, wherein the step of displaying comprises providing an indication for indicating to the user that the first transition is the expected most sensitive transition for the peptide of interest.

9. A method according to claim 7, comprising receiving from the user a second input signal for selecting the indicated at least a second transition of the plurality of candidate transitions for the peptide of interest.

10. A method according to claim 9, comprising subjecting to the SRM workflow a sample including the peptide of interest, comprising monitoring the first transition and the at least a second transition for at least one of identifying and quantifying the peptide of interest.

11. A method according to claim 10, comprising at least one of identifying and quantifying the peptide of interest based on the first transition and the at least a second transition.

12. A method according to claim 9, comprising determining separately a uniqueness of each the first transition and the at least a second transition.

13. A method according to claim 12, comprising determining an overall uniqueness of the set of transitions consisting of the first transition and the at least a second transition.

14. A method according to claim 9, wherein the first transition is the expected most sensitive transition for the peptide of interest.

15. A method according to claim 9, wherein the at least a second transition comprises a plurality of different transitions.

16. A method according to claim 10, wherein each different transition of the plurality of different transitions is indicated to the user in an iterative manner until the set of transitions consisting of the first transition and the plurality of different transitions is determined to be unique to the peptide of interest.

17. A method according to claim 7, wherein the at least a second transition is selected in an automated fashion, in dependence upon the selected first transition.

18. A method according to claim 7, wherein the first transition is a set of transitions comprising a plurality of transitions, each one of the plurality of transitions being selected based on the sensitivity criteria.

19. A system for selecting a set of transitions for a peptide of interest, the set of transitions for identifying uniquely the peptide of interest in a Selective Reaction Monitoring (SRM) workflow, the system comprising:
a controller for identifying a first SRM transition for the peptide of interest on the basis of sensitivity criteria and for identifying at least a second SRM transition for the peptide of interest on the basis of selectivity criteria, the first SRM transition relating to the formation of a first type of product ion by fragmentation of the peptide of interest and the at least a second SRM transition relating to the formation of at least a second type of product ion by fragmentation of the peptide of interest;
a detector;
a first mass separator for transmitting ions having a mass-to-charge (m/z) ratio corresponding to the peptide of interest;
an ion fragmentor for receiving the ions from the first mass separator and for fragmenting the ions to form a plurality of types of product ions; and,
a second mass separator for transmitting to the detector, sequentially in time, the first type of product ion and each different product ion type of the at least a second type of product ion.

20. A system according to claim 19, comprising a memory storage device in communication with the controller, the memory storage device for retrievably storing a database comprising information relating to a plurality of SRM transitions for at least one of unmodified and modified peptides including the peptide of interest.

21. A system according to claim 19, wherein the first mass separator, the ion fragmentor and the second mass separator are the Q1, Q2 and Q3 cells of a triple quadrupole mass spectrometer, respectively.
